(19) United States
(12) Patent Application Publication
(10) Pub. No.: US 2018/0071467 A1
(43) Pub. Date: Mar. 15, 2018

(54) SYSTEM AND METHOD FOR PORTABLE NITRIC OXIDE DELIVERY

(71) Applicant: GENO LLC, Cocoa, FL (US)

(72) Inventors: David H. Fine, Cocoa Beach, FL (US); Kurt A. Dasse, Needham, MA (US)

(73) Assignee: GENO LLC, Cocoa, FL (US)

(21) Appl. No.: 15/700,126

(22) Filed: Sep. 9, 2017

Related U.S. Application Data
(60) Provisional application No. 62/385,970, filed on Sep. 10, 2016.

(51) Int. Cl. A61M 15/00; A61M 15/08; A61B 5/021

(52) U.S. Cl. CPC A61M 15/0066; A61M 15/08; A61M 2205/8225; A61M 2202/0275; A61B 5/021

(57) ABSTRACT

An apparatus for delivering NO includes a nitric oxide-releasing agent, a reactor cartridge containing a reducing agent that coverts a nitric oxide-releasing agent to nitric oxide (NO), a portable console, and a respiratory assist device configured to deliver the NO.

### PRIORITY CLAIM

This application claims priority from U.S. Provisional Application No. 62/385,970, filed Sep. 10, 2016, which is incorporated by reference in its entirety.

### TECHNICAL FIELD

The invention relates to a system and method for portable nitric oxide delivery.

### BACKGROUND

Nitric oxide, also known as nitrosyl radical, is a free radical that is an important signaling molecule. For example, NO can cause smooth muscles in blood vessels to relax, thereby resulting in vasodilation and increased blood flow through the blood vessel. These effects can be limited to small biological regions since NO can be highly reactive with a lifetime of a few seconds and can be quickly metabolized in the body.

Some disorders or physiological conditions can be mediated by inhalation of nitric oxide. The use of low concentrations of inhaled nitric oxide can prevent, reverse, or limit the progression of disorders which can include, but are not limited to, pulmonary arterial hypertension (PAH), acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, haline membrane disease, acute pulmonary thromboembolism, heparin-protamine reactions, sepsis, asthma and status asthmaticus or hypoxia. Nitric oxide can also be used to treat chronic pulmonary hypertension, bronchopulmonary dysplasia, chronic pulmonary thromboembolism and idiopathic or primary pulmonary hypertension or chronic hypoxia.

Pulmonary arterial hypertension (PAH), is a chronic, progressive disease with an estimated incidence of 2 cases per million individuals per year and a prevalence of approximately 10 to 15 cases per million individuals. Despite the availability of a wide range of specialized therapies, mortality from PAH remains unacceptably high. Studies have provided preliminary evidence that PAH patients treated in a clinical setting, benefited from inhaled nitric oxide in reducing pulmonary pressures and pulmonary vascular resistance, without becoming tolerant to the NO. Inhaled nitric oxide is currently supplied in large tanks of compressed gas. This often limits treatment options and patient access. There is a need for a practical, safe system to treat patients with conditions such as chronic pulmonary disease in the home setting.

Respiratory assist devices (RAD's) have been shown to be effective in assisting in gas exchange for the treatment of PAH patients. The combined use of NO, using a new tank-less delivery system with a novel pump-less RAD, as an integrated system, could prove to be optimal for the treatment of patients in severe PAH as the next generation therapy. Preliminary assessments that evaluated the use of NO with oxygenators originated when tested during cardiopulmonary bypass procedures. Cardiopulmonary bypass has long been known to induce a systemic inflammatory response that contributes to clinical morbidity. The inflammatory response has been attributed to blood/biomaterial interactions with the oxygenator. Gaseous NO at low concentrations (20 ppm) has been hypothesized to elicit anti-inflammatory effects in addition to reducing pulmonary resistance. NO has been shown as able to blunt the release of markers of myocardial injury and left ventricular dysfunction during and immediately after cardiopulmonary bypass. The organ protection could be mediated, at least in part, by its anti-inflammatory properties.

Applicants have developed a fully functional wearable NO delivery system that is safe and practical for treating ambulatory patients in the home setting without the need for gas tanks. When used in connection with a pump-less, wearable respiratory assist device, the result is a novel extracorporeal, wearable, integrated NORA System to treat patients with various conditions, including severe pulmonary distress, addressing both the necessary gas exchange while simultaneously reducing pulmonary hypertension.

### SUMMARY

In general a nitric oxide delivery system can be a portably system including a disposable subsystem including a nitric oxide-releasing agent, a packaged cassette containing a reactor cartridge, a reservoir containing a nitric oxide-releasing agent and configured to release the nitric oxide-releasing agent into the reactor cartridge, a reactor cartridge containing a reducing agent that coverts a nitric oxide-releasing agent to nitric oxide (NO), a reusable subsystem including, a portable console configured to receive the cassette, and a respiratory assist device configured to deliver the NO to a subject.

In certain embodiments, the system further includes an air pump configured to provide air flow to the reactor cartridge, such that a mixture of air and NO is delivered to a patient. The system can also further include a pressure sensor.

In certain embodiments, the reservoir contains a fixed volume of liquid dinitrogen tetroxide (N₂O₄), which is in equilibrium with NO₂ gas.

In other embodiments, the system further includes a nasal cannula configured to deliver the NO.

In certain embodiments, the system can further include a nasal piece at end of cannula.

In certain embodiments, the respiratory assist device is an oxygenator. In certain embodiments, the cartridge is disposable.

The system includes an additional cartridge. In certain embodiments, the additional cartridge is disposable. In some embodiments, the first and second cartridges are identical twin cartridges. The system can also further include a third cartridge in the gas line to the patient.

The reservoir can includes glass vial. The reservoir can also include a sealed metal tube. In certain systems, the reservoir is a glass vial in a sealed metal tube.

In certain embodiments, the system is battery operated.

In certain embodiments, the nitric oxide-releasing agent is nitrogen dioxide (NO₂).

In certain embodiments, the nitric oxide-releasing agent is dinitrogen tetroxide (N₂O₄).

In certain embodiments, the nitric oxide-releasing agent is a nitrite ion (NO₂⁻). In certain embodiments, the reducing agent is ascorbic acid.

In general a method for delivering NO to a subject includes providing a nitric oxide-releasing agent, providing a reactor cartridge containing a reducing agent that coverts the nitric oxide-releasing agent to nitric oxide (NO) in a packaged cassette, inserting the packaged cassette into a console, causing the nitric oxide-releasing agent within the reactor cartridge to release a nitric oxide-releasing agent, causing a reducing agent within the reactor cartridge to convert a nitric oxide-releasing agent to nitric oxide (NO) and flowing the NO through a respiratory assist device configured to deliver the NO to a subject. The console can be a portable console, a wearable console, or a bedside console.

The method can further include applying implantable heart pressure sensors to control the amount of inhaled NO delivered to a patient in need of NO.

The method can also further include applying a pulse oximeter to control the amount of inhaled NO delivered to a patient in need of NO.

In certain methods, the nitric oxide is delivered from a fixed delivery platform. In other methods, the nitric oxide is delivered from a mobile delivery platform.

In certain methods, the nitric oxide is delivered from a bedside delivery platform.

In certain methods, liquid N₂O₄ is the source of NO.

In certain methods, the nitric oxide-releasing agent is provided from air by electrical discharge. In certain methods, the nitric oxide-releasing agent is provided from a tank of compressed gas.

In general, a method for delivering NO to a subject includes administering inhaled NO to a patient, and controlling and maintaining an optimum metabolic concentration of administered NO by feedback loop in real time.

In certain embodiments, a feedback loop is controlled by an implantable right heart pressure sensor to maintain optimum metabolic concentration.

In certain embodiments, the feedback loop can be controlled by a pulse oximeter to maintain optimum metabolic concentration.

In certain embodiments, controlling the concentration includes receiving patient data and using the data to calculate an optimum concentration to be delivered to the patient in real time.

In certain embodiments, nitric oxide is delivered from a fixed delivery platform while controlling and maintaining an optimum metabolic concentration of administered NO by feedback loop in real time. In other embodiments, nitric oxide is delivered from a mobile delivery platform and/or a bedside delivery platform while controlling and maintaining an optimum metabolic concentration of administered NO by feedback loop in real time.

Other features, objects, and advantages will be apparent from the description, drawings, and claims.

### BRIEF DESCRIPTION OF THE DRAWINGS

FIG. 1 is an embodiment of a reactor cartridge and an additional reactor cartridge.

FIG. 2 is an embodiment of a cassette containing a reactor cartridge.

FIG. 3 is an embodiment of a portable console into which a cassette is inserted.

FIG. 4 is an embodiment of the claimed system including a disposable cassette.

FIG. 5 is a schematic of the disposable subsystem and reusable subsystem.

FIG. 6 is a schematic of the disposable subsystem.

FIG. 7 is a schematic of the reusable subsystem.

FIG. 8A is an embodiment of the wearable system.

FIG. 8B is a schematic showing the wearable system.

FIG. 9 is a graph showing a method of monitoring oxygen levels and monitoring pulmonary artery pressure

### DETAILED DESCRIPTION

The administration of nitric oxide (NO) according to the claimed system and methods, allows for a novel integrated, bedside, wearable, and/or portable delivery of nitric oxide. NO can be delivered from the liquid N₂O₄, gas cylinders or tanks, or any other suitable source that generate high concentrations of NO₂ suitable for administering to a subject or patient in a therapeutic dose.

Inhaled nitric oxide (NO) is a selective and potent pulmonary vasodilator, and the therapeutic effects of inhaled NO have been shown to clinically improve pulmonary arterial pressure, pulmonary resistance, and pulmonary hemodynamics in patients with Pulmonary Arterial Hypertension (PAH) and Idiopathic Pulmonary Fibrosis (IPF). Vasodilation of the pulmonary blood vessels also can result in increased oxygenation, and NO has been shown to reduce hypoxia due to high altitude and other causes.

Inhalation of NO in the low ppm range has been shown to reduce the need for increased oxygen to maintain the same level of O2 in the blood. Inhaled nitric oxide is widely used for the treatment of a variety of related pulmonary diseases. The drug is typically given during ventilation or by means of a nasal cannula. At the present time the physician has no means of determining the needed starting dose, the optimum dose for the specific patient, the day-to-day variability of the dose, or how the NO dose should vary with time of day, the physical activity of the patient etc. The half life of NO in the body is less than a second and the vasodilatory effect occurs rapidly, typically within seconds to minutes. This rapid response opens up the possibility of monitoring and controlling the NO dose in real time, provided that there was a rapidly responding biological or mechanical marker available to act as part of a feedback loop. Two such markers are currently available, pulse oximetry that measures blood oxygen and an implantable heart pressure monitor that measures pulmonary pressures.

In general, the nitric oxide delivery system includes, the reactor cartridge that houses a reservoir containing a nitric oxide-releasing agent and a reducing agent that coverts a nitric oxide-releasing agent to nitric oxide (NO), a packaged cassette containing a reactor cartridge, a portable console configured to receive the cassette. The system delivers NO a directly to the oxygenator similar respiratory assist device, or in another embodiment, NO can be delivered via a nasal cannula, or in yet another embodiment, by both routes of administration, depending on the needs of the patient as determined by a health care provider.

For example, NO can be delivered via cannula to treat a lung condition. NO can be delivered directly to the oxygenator to the circulating blood to scavenge plasma free hemoglobin and/or treat the heart. NO can also be applied via cannula and to the oxygenator to treat any number of clinical conditions for which NO therapy is advised or deemed necessary.

There are two technologies to monitor and control the concentration of inhaled nitric oxide given to a patent so as to maximize its effect. While the technology is relevant to patients who are hospitalized, it will be of vital importance for ambulatory patients who are using a wearable inhaled NO delivery system.

Two measurements are currently used to determine whether a patient is a possible candidate for treatment with inhaled nitric oxide. They are the blood oxygen saturation as measured with a pulse oximeter and a determination of the pulmonary pressures as measured during right heart catheterization with a Swan-Ganz catheter. An important use of right heart catheterization is to measure the reduction in pulmonary pressures as a result of the inhalation of nitric oxide (NO). This is important in the treatment of PAH and IPF in that it helps determine patients who are responders if NO alleviates the higher than normal pressures.

Pulse Oximetry

Pulse oximetry is a noninvasive method for monitoring a person's oxygen saturation (SO₂). The technology typically reports its results in percentage oxygen saturation, which for a healthy person is in the 95% to 99% range. Its reading of SO₂ is not always identical to the reading of SaO₂ (arterial oxygen saturation) from arterial blood gas, but the two are correlated enough within an acceptable deviation such that the safe, convenient, noninvasive, inexpensive pulse oximetry method is valuable for measuring oxygen saturation clinically. A typical pulse oximeter utilizes an electronic processor and a pair of small light-emitting diodes (LEDs) facing a photodiode through a translucent part of the patient's body, usually a fingertip or an earlobe. One LED is red, with a wavelength of about 660 nm, and the other is in the infrared with a wavelength of about 940 nm. Absorption of light at these wavelengths differs significantly between blood loaded with oxygen and blood lacking oxygen. Oxygenated hemoglobin absorbs more infrared light and allows more red light to pass through. Deoxygenated hemoglobin allows more infrared light to pass through and absorbs more red light. It measures the changing absorbance at each of the two wavelengths, allowing it to determine the absorbance due to the pulsing arterial blood, excluding venous blood, skin, bone, muscle and fat. Pulse oximetry is particularly convenient for noninvasive continuous measurement of blood oxygen saturation. In contrast, blood gas levels must otherwise be determined in a laboratory on a drawn blood sample. Pulse oximetry is useful in any setting where a patient's oxygenation is unstable, including intensive care, operating, recovery, emergency and hospital ward settings, and for ambulatory uses for mountain climbers and athletes whose oxygen levels may decrease at high altitudes or with exercise, and for pilots in unpressurized aircraft above 10,000 feet. Pulse oximetry is small enough and light enough (the entire system including the electronics weigh only a few ounces that it can be used as a wearable sensor. A wearable pulse oximeter could be used as part of a feedback loop to control the NO dose from a wearable NO delivery system

Right-Heart Catheterization

Right heart catheterization is an invasive technology in which a special catheter (a small, hollow tube) called a pulmonary artery (PA) catheter, also called a Swan-Ganz catheter, is guided to the right side of the heart and into the pulmonary artery. This is the main artery that carries blood to the lungs. The technique is normally performed in a special catheterization facility in a hospital. The catheter allows the observation of blood flow through the heart and also measures the pressures inside the heart and lungs. The cardiac output—the amount of blood the heart pumps per minute—is also determined during a right-heart catheterization. If output from the heart is low and/or the pressures in the heart and lungs are too high, the PA catheter can be used to monitor the effects of different drugs. Right heart catheterization is also used to diagnose heart failure, a condition in which the heart muscle has become weakened, so that blood cannot be pumped efficiently, causing fluid buildup (congestion) in the blood vessels and lungs, and/or edema (swelling) in the feet, ankles, and other parts of the body. Pulmonary hypertension, where there is increased pressure within the blood vessels in the lungs, leading to difficulty breathing, can also be diagnosed by right heart catheterization.

Pulmonary Arterial Hypertension (PAH) is a debilitating disease characterized by progressive obstruction and obliteration of the pulmonary arteries leading to a rise in pulmonary vascular resistance and right ventricular failure and death. Based on pathophysiology, hemodynamics and therapeutic interventions, pulmonary hypertension according to definitions by the World Health Organization (WHO) is divided into five groups, and PAH is categorized in group one. The World Health Organization (WHO) divides pulmonary hypertension into five groups. These groups are organized based on the cause of the condition.1 Pulmonary Arterial Hypertension (PAH) comprises group number one.1 PAH is a debilitating disease characterized by progressive obstruction and obliteration of the pulmonary arteries ultimately leading to a progressive rise in pulmonary vascular resistance (PVR) and right ventricular (RV) failure and death.1-3 Pulmonary arterial hypertension can be idiopathic, hereditary, or associated with other conditions including connective tissue disease, congenital heart disease, prior anorexigen use, and HIV.

Most forms of PAH develop in adults, and in rare cases, in children; women are more likely to be affected than men. Although the mean age of patients with idiopathic PAH in the first registry created in 1981 (U.S. NIH Registry) was 36+/−15 years, PAH is now more frequently diagnosed in elderly patients, resulting in a mean age at diagnosis between 50+/−14 and 65+/−15 years in current registries. Although the female predominance is quite variable among registries, pregnancy is considered to be associated with a high rate of mortality (30-50%) in PAH patients.

Symptoms of PAH include dyspnea on exertion, fatigue, chest pain, and fainting. Chest X-ray reveals an enlarged pulmonary artery, and ECG shows right ventricular strain and hypertrophy.^{1,2,8} Echocardiography allows an estimate of pulmonary artery systolic pressure and detects cardiac disease. Right-heart catheterization is essential to establish the diagnosis: PAH is defined as mean pulmonary arterial pressure ≧25 mm Hg at rest and a normal pulmonary artery wedge pressure ≦15 mm Hg, in the absence of other disorders such as chronic thromboembolic disease or chronic respiratory diseases and/or hypoxemia.^{1,2} There is no cure for PAH; however, pharmacotherapy may be used to manage the disease and improve symptoms. As symptoms of PAH are similar to those of other diseases, diagnosis may be delayed until more advanced disease stage, when treatment is not as successful.

Idiopathic Pulmonary Fibrosis

Idiopathic pulmonary fibrosis (IPF) is a debilitating lung disorder of unknown etiology, and the most common and lethal of the idiopathic interstitial pneumonias. The disease is characterized by chronic inflammation and progressive fibrosis resulting in destruction of lung architecture, reduced lung capacity, and impaired oxygenation. The patients with IPF usually develop hypoxemia and pulmonary hypertension (PH), and PH is said to be present in up to 46% of patients with severe disease. Following diagnosis, IPF patients have a median survival time of 2 to 5 years. A number of potential risk factors have been described that contribute to the development of the disease, including age, cigarette smoking, environmental exposure (such as metal and wood dust), gastroesophageal reflux, and genetic factors.

In the pathophysiology of IPF, epithelial cell-fibroblast interaction appears to be central, where injured alveolar epithelial cells activate fibroblasts through multiple mediators and subsequent dysregulated repair. Additionally, the gas exchange and hemodynamic abnormalities IPF are said to be due to decreased synthesis of endothelium-derived potent vasodilator and anti-proliferative agent, nitric oxide (NO). In the lungs, NO is synthesized from L-arginine by the enzyme endothelial nitric oxide synthase (eNOS). The expression of eNOS in pulmonary arteries is decreased in IPF. This suggests that reduced NO synthesis may contribute to the pathogenesis of fibrosis in IPF.

Despite significant improvements made in the diagnosis of IPF, identification of a new curative treatment for IPF has not been identified. Two recently approved drugs for the treatment of IPF, pirfenidone and nintedanib, are partially effective and associated with many adverse effects. Other drug treatments including warfarin, imitanib, tanercept, intereferons, N-acetylcysteine/azathioprine/prednisolone, and ambrisentan, are either less efficacious in slowing the disease and/or are associated with severe adverse effects. In summary, despite improvements in the diagnosis and management of IPF over the last few decades, the disease continues to have a poor long-term prognosis and contributes to increased mortality in the United States.

### Nitric Oxide-Releasing Agent

In general, a nitric oxide-releasing agent such as nitrogen dioxide (NO₂), dinitrogen tetroxide (N₂O₄) and/or nitrite ions (NO₂⁻), can be converted to NO by bringing a nitric oxide-releasing agent in contact with a reducing agent.

A nitric oxide-releasing agent can be stored any suitable form, such as a liquid. The nitric oxide-releasing agent can be stored in a vessel such as a liquid vessel. A liquid vessel can contain liquid N₂O₄ for example.

A nitric oxide-releasing agent can also be contained in glass ampule. In one embodiment, a liquid vessel can contain the glass ampule, which in turn, contains a nitric oxide-releasing agent. In another embodiment, the nitric oxide-releasing agent can be contained in a liquid vessel without a glass ampule.

The nitric oxide-releasing agent can be an agent such as nitrogen tetroxide (NTO), nitrogen dioxide (NO₂), dinitrogen tetroxide (N₂O₄) or nitrite ions (NO₂⁻)

In another example, a gas including a nitric oxide-releasing agent can be passed over or through a support including a reducing agent. When the reducing agent is ascorbic acid (i.e. vitamin C), the conversion of nitrogen dioxide to nitric oxide can be quantitative at ambient temperatures.

Antioxidant

In this novel system, when a nitric oxide-releasing agent such as N₂O₄ or NTO is vaporized, it is transitioned from a liquid to gaseous NO₂, diluted with ambient air and passed through an ascorbic acid (vitamin C) cylinder where it is converted to ultra-pure NO gas for delivery to the patient. The ascorbic acid, an antioxidant, safely strips one atom of oxygen from the NO₂ to generate ultrapure NO. The benefits of this approach are substantial including: approximately 100-fold reduction in size and weight of the delivery device, which enables wearable use; the elimination of the toxic NO₂ byproduct via the antioxidant (minimizing NO₂-induced ventilation/perfusion inequality and bronchoconstriction); a substantial reduction in product cost, which expands patient access; and broadened potential applicability to numerous applications including various diseases or conditions, injuries, and improving quality of life.

In vaporizing a nitric oxide-releasing agent, a heater can be used. A heater 1 can be substantially cylindrical with a hollow body to accommodate and/or encase a reservoir containing a nitric oxide-releasing agent. A heater can be made of metal or other conductive material. It can be resistance wires. It can be a wrap around heater. It can be any convenient method to heat the nitric oxide vessel including conductance, resistance, microwave and chemical. A heater can be disposed in any suitable position to heat the nitric oxide-releasing agent.

Any suitable system can be used to deliver NO. NO can be administered by titration. Titration is a method or process of determining the concentration of a dissolved substance in terms of the smallest amount of reagent of known concentration required to bring about a given effect in reaction with a known volume of the test solution.

Delivering Inhaled NO

Inhaled NO is delivered to patients who are on ventilator and/or anesthesia machine. Patients can also receive NO by means of a nasal cannula. Typically, the concentration of NO is monitored by the console that is delivering the NO drug. Patients are typically in an intensive care under close medical supervision. The patient is slowly weaned off the drug over a period of 1 to 5 days, depending upon a variety of clinical input factors. The delivery of inhaled NO requires cylinders of compressed gas that contained NO diluted in nitrogen. This makes it difficult, if not impossible, to treat patients who are ambulatory. It has recently become possible to deliver inhaled NO from a mobile platform which is wearable and weighs about a pound. Instead of the need to have a cylinder of compressed gas typically containing about 800 ppm of NO in nitrogen, a new the technology stores the NO as liquid N₂O₄, the dimer of NO₂. During use the N₂O₄ is heated and vaporized, passed through a micron size restrictor after which the transient NO2 is reduced to NO in a cartridge containing an antioxidant.

NO can be delivered from any suitable source, including technologies which generate the NO from air by electrical discharge, gas cylinders and the use of tiny tanks of compressed gas where the NO is at a very high concentration and pulsing the high concentration to the nose as the patient takes a breath (e.g., Bellerophon). Apart from the liquid N₂O₄ technology, the other three generate high levels of NO₂.

Because of the claimed wearable technology for delivering inhaled nitric oxide, the concentration of inhaled NO delivered to the patient can be controlled by the feed back loop, using either an implantable right heart pressure sensor or a wearable pulse oximeter, or both. This makes it possible, in real time, to control the concentration of a potent drug like inhaled nitric oxide depending upon the body's need for vasodilation and/or oxygen. Because of the near instantaneous response from NO, this is the first time that the delivery of a critical drug can be controlled to maintain the optimum metabolic concentration in real time.

The electronic output of the implanted right heart pressure sensor and/or the pulse oximeter, will be sent to the computer in the wearable inhaled delivery system. The electronic signal could be sent by means of a wired cable or wirelessly using radio, electromagnetic, microwave, light or audio frequency technology. The computer in the wearable system will then take the input from the patient and use the data to calculate the optimum concentration that needs to be delivered to the patient at that moment in time. As the patient's needs change, due to physical and/or mental activity, the concentration of inhaled NO will always be optimized to the patient's needs. Physical activity, for example, like climbing stairs, will require more oxygen to be delivered to the muscles which would require additional vasodilation for the blood oxygen to remain high. Similarly, higher than desired pulmonary pressures will require additional NO to be delivered. At night, the NO level could be reduced to meet the needs of the resting patient. The same technology could be used for maintaining the optimum oxygen level for patients using a CPAP machine.

Examples of administering NO can be found, for example, in Application Ser. No. 62/266,466, Ser. Nos. 13/310,359 and 13/492,154, which is incorporated by reference herein.

Real Time Monitoring

Pulmonary arterial pressures can be monitored in a variety of ways. For example, they can be monitored by a wireless monitoring system. The wireless monitoring system is typically composed of three components: a telemetric implant (including an implantable pulmonary artery sensor), a monitoring unit, and the database management system (e.g. a Patient Electronics System) for internet-based worldwide access. The wireless monitoring system can be used to monitor the left heart (left atrium or left ventricle), right heart (right atrium or right ventricle), or both.

There are generally two categories of implants: implantable hemodynamic monitors implanted adjunct to a planned thoracic surgery and implants that are delivered percutaneously via catheter-based techniques in either the pulmonary artery (PA) or left atrium during a stand-alone procedure. The PA sensor is about the size of small paper clip and has a thin, curved wire at each end. This sensor does not require any batteries or wires.

The delivery system is a long, thin, flexible tube (catheter) that moves through the blood vessels and is designed to release the implantable sensor in the far end of the pulmonary artery.

The Patient Electronics System includes the electronics unit, antenna and pillow. Together, the components of the Patient Electronics System read the PA pressure measurements from the sensor wirelessly and then transmit the information to the doctor. The antenna is for example, paddle-shaped and is pre-assembled inside a pillow to make it easier and more comfortable for the patient to take readings. The sensor monitors the pressure in the pulmonary artery. Patients take a daily reading from home or other non-clinical locations using the Patient Electronics System which sends the information to the doctor. After analyzing the information, the doctor may make medication changes to help treat the patient's heart failure.

One example of a system used to monitor pulmonary artery pressure is the CardioMEMS™ system. The CardioMEMS HF System can be used to wirelessly measure and monitor PA pressure and heart rate in New York Heart Association (NYHA) Class III heart failure patients who have been hospitalized for heart failure in the previous year. The PA pressure and heart rate are used by doctors for heart failure management and with the goal of reducing heart failure hospitalizations. The CardioMEMS HF System is used by the doctor in the hospital or medical office setting to obtain and review PA pressure measurements. The patient uses the CardioMEMS HF System at home or other non-clinical locations to wirelessly obtain and send PA pressure and heart rate measurements to a secure database for review and evaluation by the patient's doctor.

Access to PA pressure data provides doctors with another way to better manage a patient's heart failure and potentially reduce heart failure-related hospitalizations. Reducing heart failure hospitalizations has a direct impact on a patient's well-being. In a clinical study in which 550 participants had the device implanted, there was a clinically and statistically significant reduction in heart failure-related hospitalizations for the participants whose doctors had access to PA pressure data. Additionally, there were no device or system-related complications or pressure sensor failures through six months. The system can measure pulmonary artery (PA) pressure. A pulmonary artery pressure sensor can be implanted in a pulmonary artery, and the sensor can transmit data through an electronic system. As a result, right ventricular pressure or left ventricular pressure, or both, can be evaluated.

The implanted device can collect data for pulmonary artery pressure (mPAP), systolic pulmonary artery pressure (sPAP), diastolic pulmonary artery pressure (dPAP), heart rate (HR), and/or cardia output (CO) through a sensor pressure based algorithm. The data can be collected in real time.

Use of the CardioMEMS™ in the MRI environment has been shown to be feasible and produce valuable adjunctive information. The ability to simultaneously assess volumetric and pressure responses to hemodynamic challenges has been demonstrated. Of interest is the response of the ventricular vascular coupling ratio to iNO and dobutamine. In iNO non responders, there was minimal change to ventricular vascular coupling (VVC), but patients are more responsive to changes in dobutamine.

An example of wireless monitoring is described in “A Study to Explore the Feasibility and Safety of Using Cardiomems HF System in PAH Patients,” Am. J. Respir. Crit. Care. Med. 191; 2015-A5529.

A similar wireless monitoring system can be used to monitor the right heart (right atrium or right ventricle). It is crucial to note that the two sides of the heart (left and right side) can fail independently of each other, and each event has its own causes and effects

The heart has two jobs: to collect returning, “used” blood and pump it into the lungs to be enriched with oxygen, and to take oxygen-rich blood from the lungs and pump it out to the rest of the body. The left ventricle is by far the larger of the two halves of the heart, because it does the difficult job of pumping blood out to the entire body. It draws the blood from the left lung where it has been filled with fresh oxygen. The pumping of this side of the heart sends the blood out to all the body's organs and extremities, which need the oxygen to live and work. As oxygen is depleted from the blood, it returns to the heart on the right side. The right ventricle pumps the blood back to the lungs to start the process over. Both the left and right ventricles' jobs are necessary for people to live—and either or both can be interrupted by heart failure.

Heart failure occurs when one or both sides of the heart have difficult pumping (or difficulty relaxing between pumps). This can be caused by many things, from a blood clot or heart attack to congenital factors. However, heart failure has different effects, depending on which side it strikes.

In left-sided heart failure, the heart can no longer adequately bring in fresh blood from the lung and pump it out to the body. This causes blood to back up and pool in the left lung. Shortness of breath, heaviness in the chest and difficulty breathing are common signs of left-sided heart failure.

Right-sided heart failure often occurs in response to left-sided failure. The right ventricle becomes overworked and fails in turn. If right-sided heart failure occurs on its own, blood returning from the body becomes backed up.

A PA sensor for the right heart can similarly be designed for implantation. The PA sensor for the right heart can also be configured to be about the size of small paper clip and have a thin, curved wire at each end. This sensor does not require any batteries or wires. The delivery system for the right heart can also have a long, thin, flexible tube (catheter) that moves through the blood vessels and is designed to release the implantable sensor in the far end of the pulmonary artery.

The Patient Electronics System for a right heart can also include the electronics unit, antenna and pillow. Together, the components of the Patient Electronics System read the PA pressure measurements from the sensor wirelessly and then transmit the information to the doctor. The antenna is for example, paddle-shaped and is pre-assembled inside a pillow to make it easier and more comfortable for the patient to take readings.

The sensor monitors for the right heart can also monitor the pressure in the pulmonary artery. Patients take a daily reading from home or other non-clinical locations using the Patient Electronics System which sends the information to the doctor. After analyzing the information, the doctor may make medication changes to help treat the patient's heart failure.

Nitric Oxide Delivery System

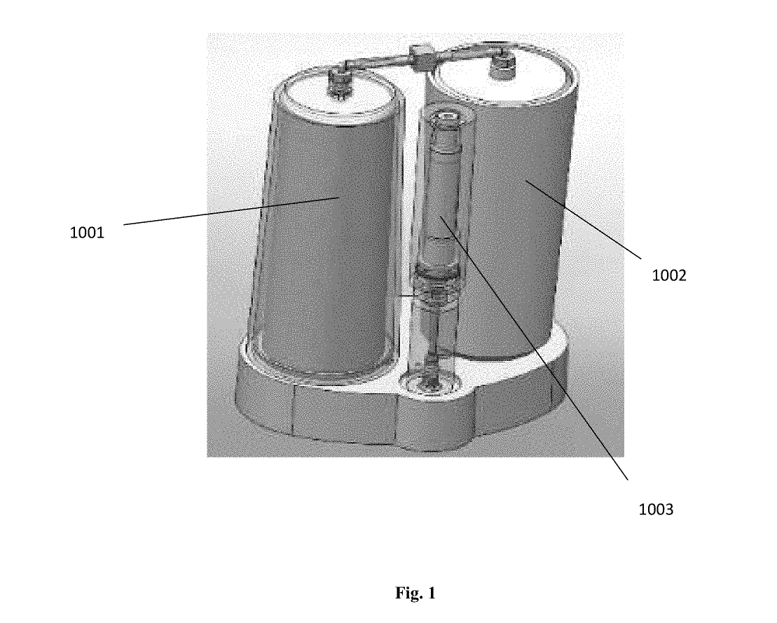

Referring to FIG. 1, the nitric oxide delivery system can include a reactor cartridge 1001 and a reservoir 1003 containing a nitric oxide-releasing agent (e.g. a glass ampule). In one embodiment, a liquid vessel can contain the glass ampule, which in turn, contains a nitric oxide-releasing agent. In other embodiments, the nitric oxide-releasing agent can be contained in a liquid vessel without a glass ampule. In certain embodiments, an additional reactor cartridge 1002 (a twin reactor cartridge), can also be used.

The nitric oxide-releasing agent is released from the reservoir into the cartridge, which contains a reducing agent (e.g. ascorbic acid) that coverts a nitric oxide-releasing agent to nitric oxide (NO).

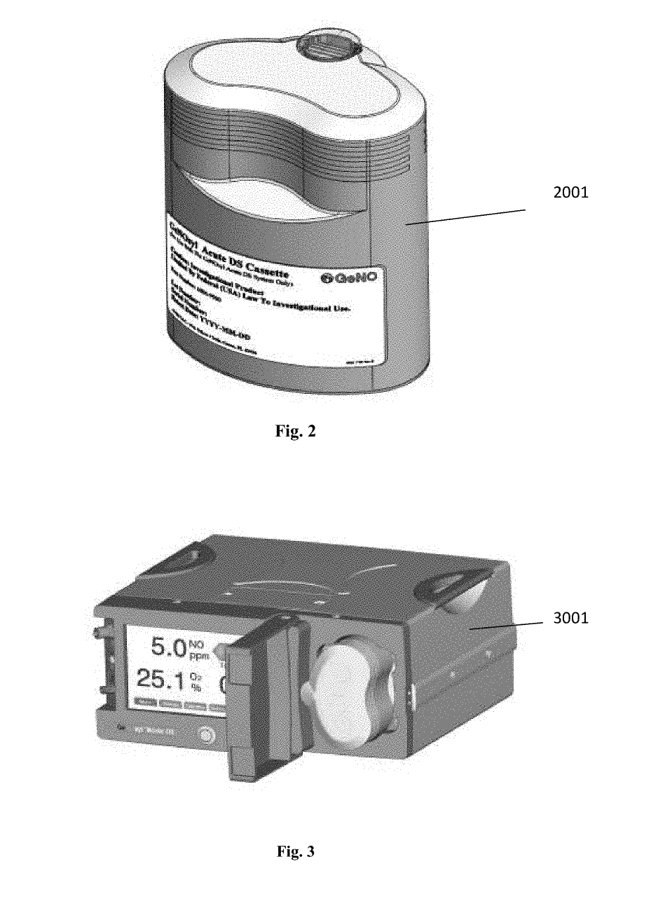

Referring to FIG. 2, a cassette 2001 can be a packaged cassette that houses at least one reactor cartridge and a reservoir for a nitric oxide releasing agent. The packaged cassette can also be configured to house two or more reactor cartridges. The cassette containing at least one reactor cartridge is configured to be inserted into a portable console. In certain embodiments, the cassette contains a reservoir, e.g., a glass vial, containing the nitric oxide-releasing agent (e.g., N₂O₄ liquid) in a doubly sealed metal tube, together with twin reactor cartridges, surrounded by an absorbent stored inside a rigid plastic housing.

Referring to FIG. 3, a cassette is configured to be inserted into a console 3001. In certain embodiments, a drug cassette is inserted into a portable console. When the cassette is activated, the reservoir (e.g. glass vial) is broken to release the nitric oxide-releasing agent (e.g. liquid N₂O₄). When the nitric oxide-releasing agent (e.g. liquid N₂O₄) is heated, it vaporizes, which produces NO₂ gas that is then forced from the cassette into the console. The NO₂ gas is passed by an internal air pump housed within the console through the first reactor cartridge within the cassette, which converts the NO₂ gas to NO. The air stream containing the therapeutic NO dose is then passed through a second, redundant reactor cartridge for added safety, and delivered to the patient through the nasal cannula. The amount of NO that is delivered to the patient is controlled under all ambient conditions. They system is designed to control the amount and concentration of the NO delivered to the patient. The system contains chemical sensors to monitor the NO concentration during drug delivery. This ensures that the NO concentration is being delivered at the set dose.

The generated nitric oxide can be delivered to a mammal, which can be a human. To facilitate delivery of the nitric oxide, a system can include a patient interface. Examples of a patient interface can include a mouth piece, nasal cannula, face mask, fully-sealed face mask or an endotracheal tube. A patient interface can be coupled to a delivery conduit. A delivery conduit can include a respiratory assist device (e.g. oxygenator), ventilator, an anesthesia machine.

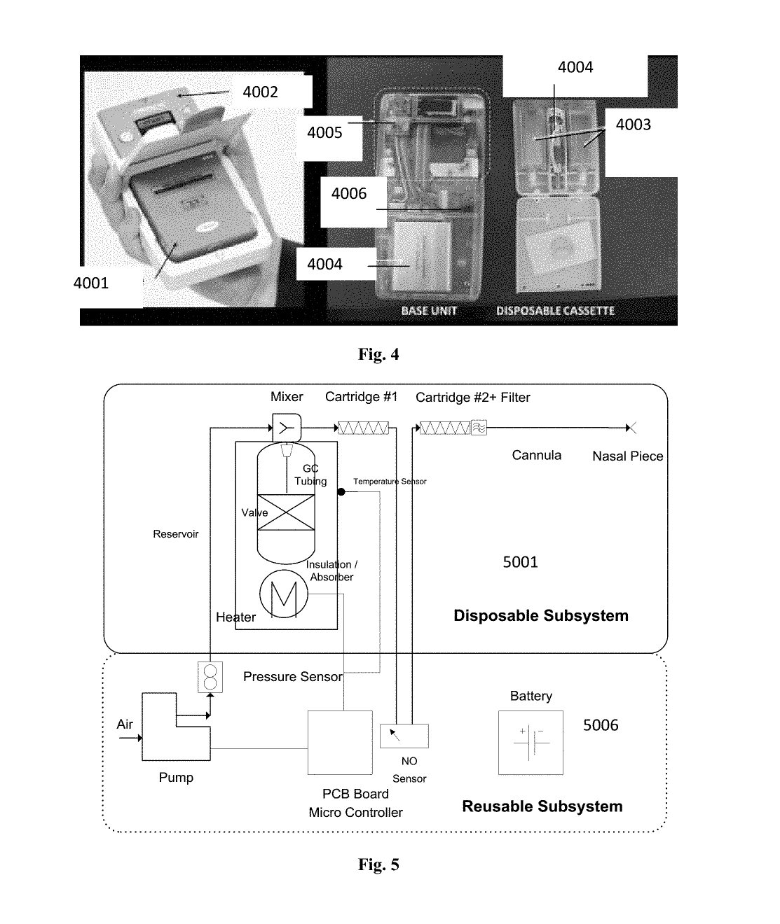

Referring to FIG. 4, a wearable system can consist of a smaller scaled cassette 4001, which can be, e.g. a disposable or single use cassette. The cassette can be configured to be inserted into a console 4002 or reusable base unit, which can be operated with a battery 4004 for wireless use. In this wearable embodiment, the cassette can also contain at least one reactor cartridge 4003 that contains an antioxidant, e.g. ascorbic acid, and a reservoir 4004 containing a nitric oxide-releasing agent, which reservoir can be a NTO liquid vessel and restrictor assembly.

Each cartridge is comprised of a blend of antioxidants, polymers and silica. As NO₂ gas passes through the reactor cartridge, a single oxygen atom is stripped away from each NO₂ molecule to create NO. A constant flow of NO from the cassette passes through a low pressure drop cartridge where any residual NO₂ gas that is formed in the sample lines is removed before delivery. The low pressure drop cartridge is also designed to mix the gas so that the NO concentration inhaled by the patient remains constant during the breathing cycle.

When the cassette is activated, the reservoir (e.g. NTO liquid vessel and restrictor assembly) releases the nitric oxide-releasing agent (e.g. liquid N₂O₄). When the nitric oxide-releasing agent (e.g. liquid N₂O₄) is heated, it vaporizes, and produces NO₂ gas as it is passes through a micron bore restrictor. The transient NO₂ is mixed with air from an internal pump and the NO₂ in air is then converted to NO by passing it through an cartridge that contains a reducing agent. A second redundant cartridge is also used. The air stream containing the therapeutic NO dose is then delivered to the patient, e.g., through the nasal cannula. Just before the patient a third cartridge is used to remove any NO2 that was formed in the gas lines to the patient. The amount of NO that is delivered to the patient is controlled under all ambient conditions. They system includes an electronic control card 4006, which is designed to control the amount and concentration of the NO delivered to the patient. The system contains chemical sensors to monitor the NO concentration during drug delivery. This ensures that the NO concentration is being delivered at the set dose.

Referring to FIG. 5, the wearable system consists of a disposable subsystem 5001 and the reusable subsystem 5006. The disposable subsystem includes the cassette, which houses the reactor cartridge(s) and the reservoir assembly containing the nitric oxide releasing agent (e.g. NTO liquid vessel, restrictor assembly and a heater designed to heat and/or vaporize the nitric-oxide releasing agent. The reservoir assembly can include a valve, which when activated, can release the nitric-oxide releasing agent into the cartridge.

The reusable subsystem includes the base unit or portable console, the electronic control card (PCB board or micro controller) and gas flow assembly including a pump, pressure sensor and a battery.

In certain embodiments, the system has a reservoir with a fixed volume of liquid dinitrogen tetroxide (N₂O₄), which is in equilibrium with NO₂ gas. The system generates and delivers a fixed dose of NO gas in a non-hypoxic, breathable gas to patients. Micron bore tubing used in gas chromatography a constant flow of NO₂ from the reservoir to a mixing chamber with air flowing at approximately 1 L/min. A miniature pump is used to provide air flow at the specified flow. A gas mixture composed of air and NO₂ flows through the first and second GeNO cartridges. The output of the second cartridge (air and NO) is conveyed to the patient using flexible tubing and a nasal cannula. A micro bacterial filter is also used at the end of the second cartridge assembly. In order to keep NO₂ flow constant, the system keeps the temperature constant by using a heating element in combination with a temperature sensor and a closed loop control system. This ensures a constant concentration of NO in the air mixture delivered to the patient.

In certain embodiments, the system is battery operated and fully instrumented for continuous operation and temperature control. The system provides power from a single rechargeable battery pack. For example, an intelligent 110V/220V battery charger/power supply unit can be used for both recharging the battery and powering the system to guarantee continuous operation. A back-up battery can also be used to alert the user to power the system with an external power source.

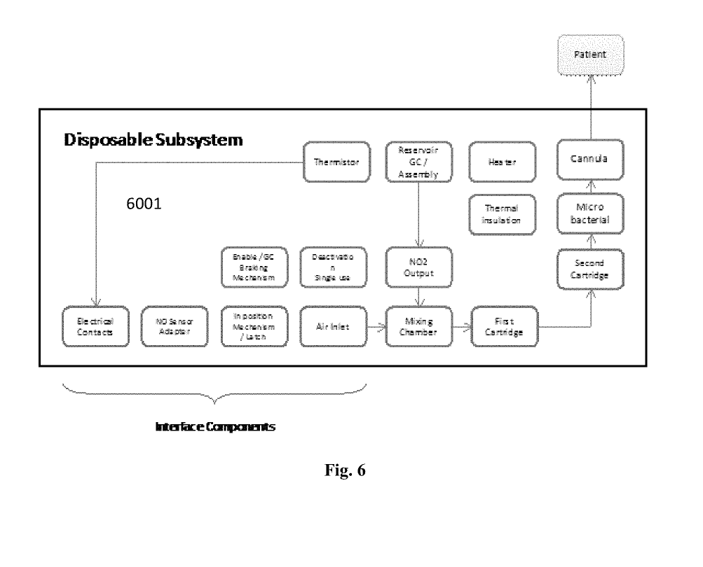

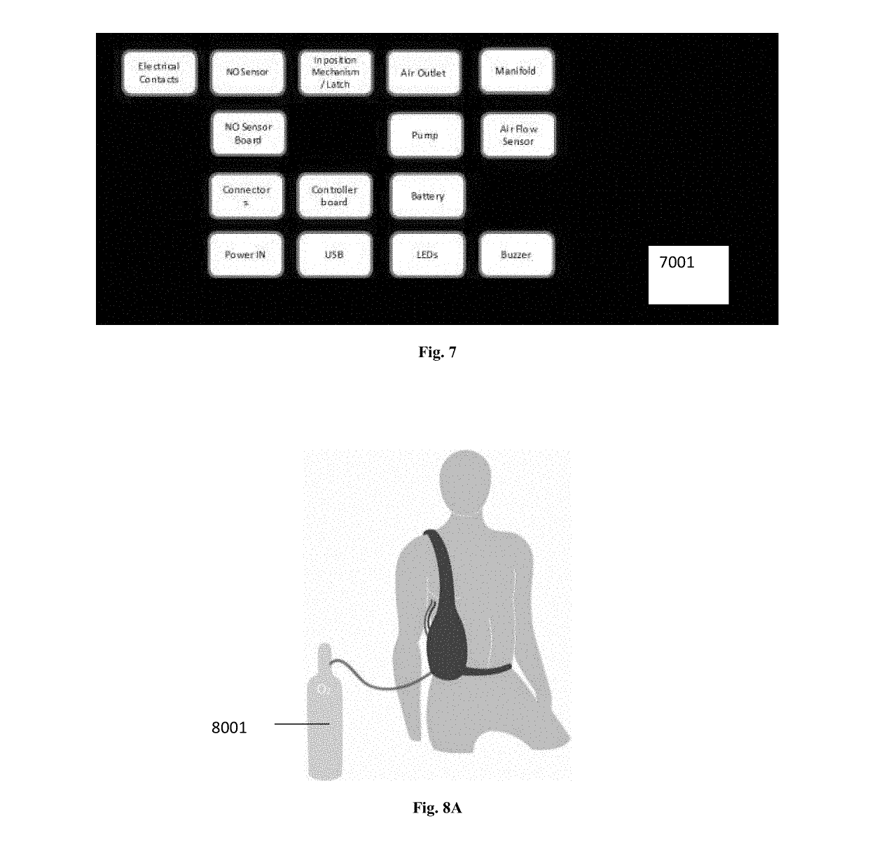

Referring to FIGS. 6 and 7, an interface provides the basic electrical and mechanical connections in order to guarantee the functionality between these two subsystems or modules. In certain embodiments, the disposable subsystem 6001 contains the reservoir with N₂O₄ liquid in equilibrium with NO₂ gas, heating element, temperature sensor, insulation/absorber, and a GC column assembly to provide the required NO₂ flow, with a mixer chamber that connects to the pump air outlet, GeNO cartridges, and a connection for the nasal cannula assembly to deliver NO to the patient. The disposable unit is for usually, but not always for single-use. The reservoir is sized to provide 12, 24, 48 or 60 hours of NO to the patient. When the user removes the disposable unit, it is permanently disabled to prevent reuse. The disposable also provides a visual indication that it has been used and should be discarded. In certain embodiments, the reusable subsystem (FIG. 7) provides the air pump, battery, micro-controller, power management, NO sensor, pressure sensor (to measure air flow), atmospheric pressure sensor, sensors interface, user interface (indicators/alarms) and electronics circuits. The interface between these two modules provides the following: a temperature sensor connector, an electrical connection for the heater, air connection, connections for NO sensor, in position sensor, and latch mechanism.

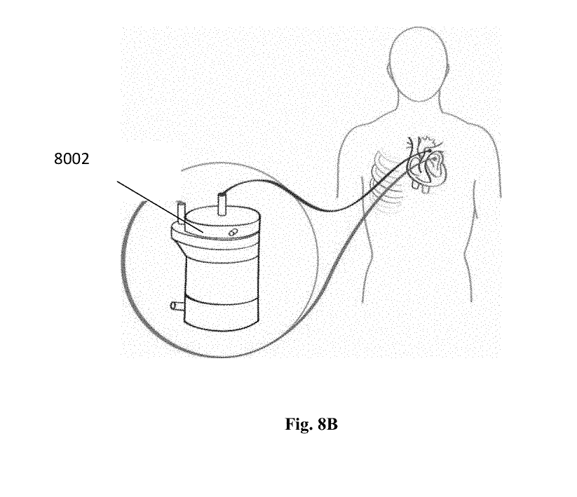

Referring to FIGS. 8A and 8B, a respiratory assist device (RAD) 8001, is cannulated from the pulmonary artery to the left atrium (parallel to the patient's lungs) to unload the lungs and right ventricle simultaneously. Due to the integrated compliance as well as the minimal flow resistance of 7.5 mmHg at maximum flow, the RAD can be used without the need for a blood pump, energy supply or controller. The blood flow across RAD is dependent on the pressure difference between Pulmonary Artery and Left Atrium (PA-LA), allowing for higher blood flows in patients with severe pulmonary hypertension than previously possible. The elasticity of the compliance can be adjusted, while the RAD is in use. The ability to adjust the compliance affects the flow resistance and hence the blood flow across the device. This makes this RAD the only available lung assist device with adjustable flow resistance.

Referring to FIG. 8B, an integrated RAD 8002 uses a unique technique to integrate numerous elastic elements into the fiber bundle of a gas exchanger, to create a compliance comparable to the physiological properties of the native lungs. These elements also guide the blood flow across the fiber bundle, creating very efficient flow conditions. During systole, the heart ejects blood and increases the pressure in the pulmonary circulation. This causes the elastic elements inside the RAD to collapse and creates more space between the gas exchanging fibers which reduces the flow resistance. During diastole, the blood pressure is reduced and the elastic elements form back into the original shape, ejecting the blood towards the left atrium. Due to the defined and numerically optimized arrangement of different elastic elements within the RAD, as well as the ability to create motion inside the fiber bundle, in this device, there are no areas of stagnation that could lead to thrombus formation. This is crucial since thrombus formation is one of the major limitations of current long-term lung support systems.

For gas exchange, an integrated RAD can use hollow fibers made of Polymethylpentene (OXYPLUS®). These fibers are used in commercial lung support systems and provide sufficient gas exchange for several weeks. The gas exchange efficiency of the RAD is comparable to those of commercially available oxygenator systems, while at the same time having a significantly lower pressure loss due to the unique compliance function.

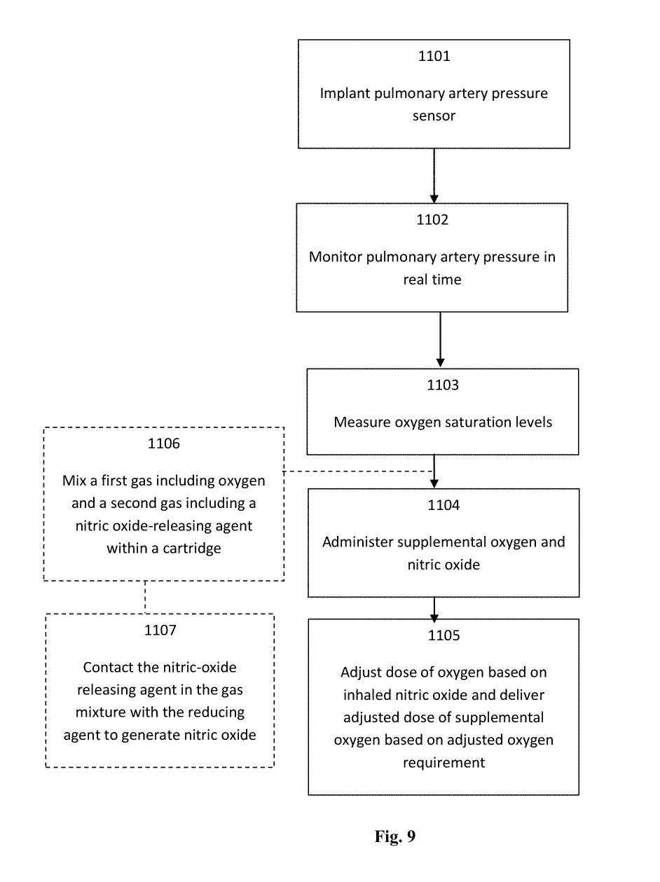

FIG. 9 shows an embodiment of the invention. The method includes implanting a pulmonary artery pressure sensor (1101), monitoring pulmonary artery pressure in real time (1102), measuring oxygen levels in a patient (1103), administer supplemental oxygen and nitric oxide (1104), and adjusting dose of oxygen based on inhaled nitric oxide and deliver adjusted dose of supplemental oxygen based on adjusted oxygen requirement (1105). In certain embodiments, the method can optionally include mixing a first gas including oxygen gas and a second gas including a nitric-oxide releasing agent within a cartridge (1106) and then contacting the nitric oxide-releasing agent with the reducing agent to generate nitric oxide (1107). The method can further include determining a first oxygen requirement based on a patient's condition or disease state, for example. Upon determining an oxygen requirement, a clinician such as a physician or other professional or person operating in a health care capacity, can then adjust the dose of oxygen in real time to a second dose based on the inhaled nitric oxide. The clinician can determine a reduced oxygen requirement in view of the inhaled nitric oxide, either before or after the dose of oxygen is adjusted to a second dose or titrated until a target level of oxygen is reached. After a reduced oxygen requirement is determined or adjusted, a clinician can deliver a dose of supplemental oxygen based on the reduced oxygen requirement and the gas mixture including nitric oxide.

Constant NO injection into the breathing circuit can be a simple and viable technique as long as a receptacle is both a mixer with sufficient volume and can remove NO₂ from the circuit or can convert the NO₂ back into NO.

Various Embodiments

In certain embodiments, after use, an absorbent can eliminate residual N₂O₄ liquid remaining in the cassette. Twin-cartridges in the cassette serve as reactors that convert the NO₂ gas into therapeutic NO. Each of the twin cartridges can be cylindrical-shaped and about the size and shape of a slightly elongated D-cell battery. The reactor cartridges are designed with extra capacity to convert significantly more than the content of the vial of liquid N₂O₄ into NO. While only one reactor cartridge is needed for a therapeutic application, but a second cartridge (which can be an identical cartridge) can be incorporated for redundancy enhanced safety.

A cartridge can include an inlet and an outlet. A cartridge can convert a nitric oxide-releasing agent to nitric oxide (NO). A cartridge can include a reducing agent or a combination of reducing agents. A number of reducing agents can be used depending on the activities and properties as determined by a person of skill in the art. In some embodiments, a reducing agent can include a hydroquinone, glutathione, and/or one or more reduced metal salts such as Fe(II), Mo(VI), NaI, Ti(III) or Cr(III), thiols, or NO₂⁻. A reducing agent can include 3,4 dihydroxy-cyclobutene-dione, maleic acid, croconic acid, dihydroxy-fumaric acid, tetra-hydroxy-quinone, p-toluene-sulfonic acid, tricholor-acetic acid, mandelic acid, 2-fluoro-mandelic acid, or 2,3,5,6-tetrafluoro-mandelic acid. A reducing agent can be safe (i.e., non-toxic and/or non-caustic) for inhalation by a mammal, for example, a human. A reducing agent can be an antioxidant. An antioxidant can include any number of common antioxidants, including ascorbic acid, alpha tocopherol, and/or gamma tocopherol. A reducing agent can include a salt, ester, anhydride, crystalline form, or amorphous form of any of the reducing agents listed above. A reducing agent can be used dry or wet. For example, a reducing agent can be in solution. A reducing agent can be at different concentrations in a solution. Solutions of the reducing agent can be saturated or unsaturated. While a reducing agent in organic solutions can be used, a reducing agent in an aqueous solution is preferred. A solution including a reducing agent and an alcohol (e.g. methanol, ethanol, propanol, isopropanol, etc.) can also be used.

A cartridge can include a support. A support can be any material that has at least one solid or non-fluid surface (e.g. a gel). It can be advantageous to have a support that has at least one surface with a large surface area. In preferred embodiments, the support can be porous or permeable. One example of a support can be surface-active material, for example, a material with a large surface area that is capable of retaining water or absorbing moisture. Specific examples of surface active materials can include silica gel or cotton. The term “surface-active material” denotes that the material supports an active agent on its surface. The surface active material is needed for a second reason, namely to trap out and remove the organic and inorganic by products of the reaction with the transient NO2. The material must also be capable of trapping out and removing water vapor and nitric and nitrous acids which are formed by the reaction of NO and NO2 with moisture.

A support can include a reducing agent. Said another way, a reducing agent can be part of a support. For example, a reducing agent can be present on a surface of a support. One way this can be achieved can be to coat a support, at least in part, with a reducing agent. In some cases, a system can be coated with a solution including a reducing agent. Preferably, a system can employ a surface-active material coated with an aqueous solution of antioxidant as a simple and effective mechanism for making the conversion. Generation of NO from a nitric oxide-releasing agent performed using a support with a reducing agent can be the most effective method, but a reducing agent alone can also be used to convert nitric oxide-releasing agent to NO.

In some circumstances, a support can be a matrix or a polymer, more specifically, a hydrophilic polymer. A support can be mixed with a solution of the reducing agent. The solution of reducing agent can be stirred and strained with the support and then drained. The moist support-reducing agent mixture can be dried to obtain the proper level of moisture. Following drying, the support-reducing agent mixture may still be moist or may be dried completely. Drying can occur using a heating device, for example, an oven or autoclave, or can occur by air drying.

In one embodiment of a cartridge for generating NO by converting a nitric oxide-releasing agent to NO, a cartridge can include an inlet and an outlet. A cartridge can be inserted into and removed from an apparatus, platform or system. Preferably, a cartridge is replaceable in the apparatus, platform or system, and more preferably, a cartridge can be disposable. Screen and glass wool can be located at either or both of the inlet and the outlet. The remainder of the cartridge can include a support. In a preferred embodiment, a cartridge can be filled with a surface-active material. The surface-active material can be soaked with a saturated solution of antioxidant in water to coat the surface-active material. The screen and glass wool can also be soaked with the saturated solution of antioxidant in water before being inserted into the cartridge.

In general, a process for converting a nitric oxide-releasing agent to NO can include passing a gas including a nitric oxide-releasing agent into the inlet. The gas can be communicated to the outlet and into contact with a reducing agent. In a preferred embodiment, the gas can be fluidly communicated to the outlet 110 through the surface-active material coated with a reducing agent. As long as the surface-active material remains moist and the reducing agent has not been used up in the conversion, the general process can be effective at converting a nitric oxide-releasing agent to NO at ambient temperature.

The inlet may receive the gas including a nitric oxide-releasing agent from a gas pump that fluidly communicates the gas over a diffusion tube or a permeation cell. The inlet also may receive the gas including a nitric oxide-releasing agent, for example, from a pressurized bottle of a nitric oxide-releasing agent. A pressurized bottle may also be referred to as a tank. The inlet also may receive a gas including a nitric oxide-releasing agent can be NO₂ gas in nitrogen (N₂), air, or oxygen (O₂). A wide variety of flow rates and NO₂ concentrations have been successfully tested, ranging from only a few ml per minute to flow rates of up to 5,000 ml per minute.

The conversion of a nitric oxide-releasing agent to NO can occur over a wide range of concentrations of a nitric oxide-releasing agent. For example, experiments have been carried out at concentrations in air of from about 2 ppm NO₂ to 100 ppm NO₂, and even to over 1000 ppm NO₂. In one example, a cartridge that was approximately 6 inches long and had a diameter of 1.5-inches was packed with silica gel that had first been soaked in a saturated aqueous solution of ascorbic acid. The moist silica gel was prepared using ascorbic acid designated as A.C.S reagent grade 99.1% pure from Aldrich Chemical Company and silica gel from Fischer Scientific International, Inc., designated as S8 32-1, 40 of Grade of 35 to 70 sized mesh. Other sizes of silica gel can also be effective. For example, silica gel having an eighth-inch diameter can also work.

In another example, silica gel was moistened with a saturated solution of ascorbic acid that had been prepared by mixing 35% by weight ascorbic acid in water, stirring, and straining the water/ascorbic acid mixture through the silica gel, followed by draining. The conversion of NO₂ to NO can proceed well when the support including the reducing agent, for example, silica gel coated with ascorbic acid, is moist. In a specific example, a cartridge filled with the wet silica gel/ascorbic acid was able to convert 1000 ppm of NO₂ in air to NO at a flow rate of 150 ml per minute, quantitatively, non-stop for over 12 days.

A cartridge can be used for inhalation therapy. In addition to converting a nitric oxide-releasing agent to nitric oxide to be delivered during inhalation therapy, a cartridge can remove any NO₂ that chemically forms during inhalation therapy (e.g., nitric oxide that is oxidized to form nitrogen dioxide). In one such example, a cartridge can be used as a NO₂ scrubber for NO inhalation therapy that delivers NO from a pressurized bottle source. A cartridge may be used to help ensure that no harmful levels of NO₂ are inadvertently inhaled by the patient.

In addition, a cartridge may be used to supplement or replace some or all of the safety devices used during inhalation therapy in conventional NO inhalation therapy. For example, one type of safety device can warn of the presence of NO₂ in a gas when the concentration of NO₂ exceeds a preset or predetermined limit, usually 1 part per million or greater of NO₂. Such a safety device may be unnecessary when a cartridge is positioned in a NO delivery system just prior to the patient breathing the NO laden gas. A cartridge can convert any NO₂ to NO just prior to the patient breathing the NO laden gas, making a device to warn of the presence of NO₂ in gas unnecessary.

Furthermore, a cartridge placed near the exit of inhalation equipment, gas lines or gas tubing can also reduce or eliminate problems associated with formation of NO₂ that occur due to transit times in the equipment, lines or tubing. As such, use of a cartridge can reduce or eliminate the need to ensure the rapid transit of the gas through the gas plumbing lines that is needed in conventional applications. Also, a cartridge can allow the NO gas to be used with gas balloons to control the total gas flow to the patient.

Alternatively or additionally, a NO₂ removal cartridge can be inserted just before the attachment of the delivery system to the patient to further enhance safety and help ensure that all traces of the toxic NO₂ have been removed. The NO₂ removal cartridge may be a cartridge used to remove any trace amounts of NO₂. Alternatively, the NO₂ removal cartridge can include heat-activated alumina. A cartridge with heat-activated alumina, such as supplied by Fisher Scientific International, Inc., designated as ASOS-212, of 8-14 sized mesh can be effective at removing low levels of NO₂ from an air or oxygen stream, and yet, can allow NO gas to pass through without loss. Activated alumina, and other high surface area materials like it, can be used to scrub NO₂ from a NO inhalation line.

In another example, a cartridge can be used to generate NO for therapeutic gas delivery. Because of the effectiveness of a cartridge in converting nitric oxide-releasing agents to NO, nitrogen dioxide (gaseous or liquid) or dinitrogen tetroxide can be used as the source of the NO. When nitrogen dioxide or dinitrogen tetroxide is used as a source for generation of NO, there may be no need for a pressurized gas bottle to provide NO gas to the delivery system. By eliminating the need for a pressurized gas bottle to provide NO, the delivery system may be simplified as compared with a conventional apparatus that is used to deliver NO gas to a patient from a pressurized gas bottle of NO gas. A NO delivery system that does not use pressurized gas bottles may be more portable than conventional systems that rely on pressurized gas bottles.

In some delivery systems, the amount of nitric oxide-releasing agent in a gas can be approximately equivalent to the amount of nitric oxide to be delivered to a patient. For example, if a therapeutic dose of 20 ppm of nitric oxide is to be delivered to a patient, a gas including 20 ppm of a nitric oxide-releasing agent (e.g., NO₂) can be released from a gas bottle or a diffusion tube. The gas including 20 ppm of a nitric oxide-releasing agent can be passed through one or more cartridges to convert the 20 ppm of nitric oxide-releasing agent to 20 ppm of nitric oxide for delivery to the patient. However, in other delivery systems, the amount of nitric oxide-releasing agent in a gas can be greater than the amount of nitric oxide to be delivered to a patient. For example, a gas including 800 ppm of a nitric oxide-releasing agent can be released from a gas bottle or a diffusion tube. The gas including 800 ppm of a nitric oxide-releasing agent can be passed through one or more cartridges to convert the 800 ppm of nitric oxide-releasing agent to 800 ppm of nitric oxide. The gas including 800 ppm of nitric oxide can then be diluted in a gas including oxygen (e.g., air) to obtain a gas mixture with 20 ppm of nitric oxide for delivery to a patient. Traditionally, the mixing of a gas including nitric oxide with a gas including oxygen to dilute the concentration of nitric oxide has occurred in a line or tube of the delivery system. The mixing of a gas including nitric oxide with a gas including oxygen can cause problems because nitrogen dioxide can form. To avoid this problem, two approaches have been used. First, the mixing of the gases can be performed in a line or tube immediately prior to the patient interface, to minimize the time nitric oxide is exposed to oxygen, and consequently, reduce the nitrogen dioxide formation. Second, a cartridge can be placed at a position downstream of the point in the line or tubing where the mixing of the gases occurs, in order to convert any nitrogen dioxide formed back to nitric oxide.

While these approaches can minimize the nitrogen dioxide levels in a gas delivered to a patient, these approaches have some drawbacks. Significantly, both of these approaches mix a gas including nitric oxide with a gas including oxygen in a line or tubing of the system. One problem can be that lines and tubing in a gas delivery system can have a limited volume, which can constrain the level of mixing. Further, a gas in lines and tubing of a gas delivery system can experience variations in pressure and flow rates. Variations in pressure and flow rates can lead to an unequal distribution of the amount each gas in a mixture throughout a delivery system. Moreover, variations in pressure and flow rates can lead to variations in the amount of time nitric oxide is exposed to oxygen within a gas mixture. One notable example of this arises with the use of a ventilator, which pulses gas through a delivery system. Because of the variations in pressure, variations in flow rates and/or the limited volume of the lines or tubing where the gases are mixed, a mixture of the gases can be inconsistent, leading to variation in the amount of nitric oxide, nitrogen dioxide, nitric oxide-releasing agent and/or oxygen between any two points in a delivery system.

To address these problems, a mixing chamber can also be used to mix a first gas and a second gas. A first gas can include oxygen; more specifically, a first gas can be air. A second gas can include a nitric oxide-releasing agent and/or nitric oxide. A first gas and a second gas can be mixed within a mixing chamber to form a gas mixture. The mixing can be an active mixing performed by a mixer. For example, a mixer can be a moving support. The mixing within a cartridge or mixing chamber can also be a passive mixing, for example, the result of diffusion.

### NO Delivery

A cartridge can be coupled to a gas conduit. A first gas including oxygen can be communicated through a gas conduit to the cartridge. The communication of the first gas through the gas conduit can be continuous or it can be intermittent. For instance, communicating the first gas intermittently can include communicating the first gas through the gas conduit in one or more pulses. Intermittent communication of the first gas through gas conduit can be performed using a gas bag, a pump, a hand pump, an anesthesia machine or a ventilator.

A gas conduit can include a gas source. A gas source can include a gas bottle, a gas tank, a permeation cell or a diffusion tube.

Nitric oxide delivery systems including a gas bottle, a gas tank, a permeation cell or a diffusion tube are described, for example, in U.S. Pat. Nos. 7,560,076 and 7,618,594, each of which are incorporated by reference in its entirety. Alternatively, a gas source can include a reservoir and restrictor, as described in U.S. patent application Ser. Nos. 12/951,811, 13/017,768 and 13/094,535, each of which is incorporated by reference in its entirety.

A gas source can include a pressure vessel, as described in U.S. patent application Ser. No. 13/492,154, which is incorporated by reference in its entirety. A gas conduit can also include one or more additional cartridges.

Additional components including one or more sensors for detecting nitric oxide levels, one or more sensors for detecting nitrogen dioxide levels, one or more sensor for detecting oxygen levels, one or more humidifiers, valves, tubing or lines, a pressure regulator, flow regulator, a calibration system and/or filters can also be included in a gas conduit.

A second gas can also be communicated to a cartridge. A second gas can be supplied into a gas conduit. Preferably, a second gas can be supplied into a gas conduit immediately prior to a cartridge. A second gas can be supplied into a gas conduit via a second gas conduit, which can join or be coupled to the gas conduit. Once a second gas is supplied into a gas conduit, both the first gas and the second gas can be communicated in the inlet of a cartridge for mixing. Alternatively, a second gas can be supplied at a cartridge. For example, a second gas can be supplied directly into the inlet of a cartridge.

Once a first gas and a second gas are within a cartridge, a first gas and a second gas can mix to form a gas mixture including oxygen and one or more of nitric oxide, a nitric oxide-releasing agent (which can be nitrogen dioxide) and nitrogen dioxide. The gas mixture can contact a reducing agent, which can be on a support within the cartridge. The reducing agent can convert nitric oxide-releasing agent and/or nitrogen dioxide in the gas mixture to nitric oxide.

The gas mixture including nitric oxide can then be delivered to a mammal, most preferably, a human patient. The concentration of nitric oxide in a gas mixture can be at least 0.01 ppm, at least 0.05 ppm, at least 0.1 ppm, at least 0.5 ppm, at least 1 ppm, at least 1.5 ppm, at least 2 ppm or at least 5 ppm. The concentration of nitric oxide in a gas mixture can be at most 100 ppm, at most 80 ppm, at most 60 ppm, at most 40 ppm, at most 25 ppm, at most 20 ppm, at most 10 ppm, at most 5 ppm or at most 2 ppm.

### Delivery Conduit

Delivering the gas mixture including nitric oxide from the cartridge to the mammal can include passing the gas mixture through a delivery conduit. A delivery conduit can be located between the cartridge and a patient interface. In some embodiments, a delivery conduit can be coupled to the outlet of a cartridge and/or coupled to the patient interface. A delivery conduit can include additional components, for example, a humidifier or one or more additional cartridges.

Delivery of a gas mixture can include continuously providing the gas mixture to the mammal. When the delivery of the gas mixture includes continuously providing the gas mixture to the mammal, the volume of the cartridge can be greater than the volume of the delivery conduit. The larger volume of the cartridge can help to ensure that the gas mixture is being thoroughly mixed prior to delivery. Generally, more complete mixing can occur as the ratio of the volume of the cartridge to the volume of the delivery conduit increases. A preferable level of mixing can occur when the volume of the cartridge is at least twice the volume of the delivery conduit. The volume of the cartridge can also be at least 1.5 times, at least 3 times, at least 4 times or at least 5 times the volume of the delivery conduit.

When the volume of the cartridge is greater than the volume of the delivery conduit or the volume of gas mixture in the delivery conduit, the gas mixture may not go directly from the cartridge to the mammal, but instead, can be delayed in the cartridge or delivery conduit. It is this delay that can provide the time needed to mix the gas so that the NO concentration remains constant within a breath.

This delay can result in the storage of the gas mixture in the cartridge. The gas mixture can be stored in the cartridge for a predetermined period of time. The predetermined period of time can be at least 1 second, at least 2 seconds, at least 6 seconds, at least 10 seconds, at least 20 seconds, at least 30 seconds or at least 1 minute.

The mixing that occurs due to the delay of the gas mixture (i.e. storage of the gas mixture in a cartridge) can be so effective that the intra-breath variation can be identical to what could be achieved under ideal conditions when premixed gas was provided. This can be referred to as “perfect mixing.” For continuous delivery, this can mean that the concentration of nitric oxide in the gas mixture delivered to a mammal remains constant over a period of time (e.g. at least 1 min, at least 2 min, at least 5 min, at least 10 min or at least 30 min). For a concentration to remain constant, the concentration can remain with a range of at most ±10%, at most ±5%, or at most ±2% of a desired concentration for delivery.

Delivery of the gas mixture can include intermittently providing the gas mixture to the mammal. Intermittent delivery of a gas mixture can be the result of intermittent communication of a first or second gas into the system. Said another way, intermittent communication of a first or second gas through a gas conduit can result in an increased area of pressure, which can traverse into the cartridge causing intermittent communication of the gas mixture. Intermittent delivery can be performed using a gas bag, a pump, a hand pump, an anesthesia machine or a ventilator.

The intermittent delivery can include an on-period, when the gas mixture is delivered to a patient, and an off-period, when the gas mixture is not delivered to a patient. Intermittent delivery can include delivering one or more pules of the gas mixture.

An on-period or a pulse can last for a few seconds up to as long as several minutes. In one embodiment, an on-period or a pulse can last for 1, 5, 10, 15, 20, 25, 30, 35, 40, 45, 50, 55 or 60 seconds. In another embodiment, the on-period or a pulse can last for 1, 2, 3, 4 or 5 minutes. In a preferred embodiment, an on-period or a pulse can last for 0.5-10 seconds, most preferably 1-6 seconds.

Intermittent delivery can include a plurality of on-periods or pulses. For example, intermittent delivery can include at least 1, at least 2, at least 5, at least 10, at least 50, at least 100 or at least 1000 on-periods or pulses.

The timing and duration of each on-period or pulse of the gas mixture can be pre-determined. Said another way, the gas mixture can be delivered to a patient in a pre-determined delivery sequence of one or more on-periods or pulses. This can be achieved using an anesthesia machine or a ventilator, for example.

When the delivery of the gas mixture includes intermittently providing the gas mixture to the mammal, the volume of the cartridge can be greater than the volume of the gas mixture in a pulse or on-period. The larger volume of the cartridge can help to ensure that the gas mixture is being thoroughly mixed prior to delivery. Generally, more complete mixing can occur as the ratio of the volume of the cartridge to the volume of the gas mixture in a pulse or on-period delivered to a mammal increases. A preferable level of mixing can occur when the volume of the cartridge is at least twice the volume of the gas mixture in a pulse or on-period. The volume of the cartridge can also be at least 1.5 times, at least 3 times, at least 4 times or at least 5 times the volume of the gas mixture in a pulse or on-period.

When the volume of the cartridge is greater than the volume of the volume of the gas mixture in a pulse or on-period, the gas mixture may not go directly from the cartridge to the mammal, but instead, can be delayed in the cartridge or delivery conduit for one or more pulses or on-periods. It is this delay that can provide the time needed to mix the gas so that the NO concentration remains constant between delivered pulses or on-periods.

In addition to storage as a result of off-periods, the delay caused by the differing volumes can result in the storage of the gas mixture in the cartridge. The gas mixture can be stored in the cartridge for a predetermined period of time. The predetermined period of time can be during or between pulses or on-periods. The predetermined period of time can be at least 1 second, at least 2 seconds, at least 6 seconds, at least 10 seconds, at least 20 seconds, at least 30 seconds or at least 1 minute.

The mixing that occurs due to the delay of the gas mixture (i.e. storage of the gas mixture in a cartridge) can be so effective that the intra-breath variation can be identical to what could be achieved under ideal conditions when premixed gas was provided. Intermittent delivery an include providing the gas mixture for two or more pulses or on-periods. Using intermittent delivery, the concentration of nitric oxide in each pulse or on-period can vary by less than 10%, by less than 5%, or by less than 2%. In other words, the variation between the concentration of nitric oxide in a first pulse and the concentration of nitric oxide in a second pulse is less than 10% (or less than 5% or 2%) of the concentration of nitric oxide in the first pulse. In another embodiment, using intermittent delivery, the concentration of nitric oxide in each pulse or on-period can vary by less than 10 ppm, less than 5 ppm, less than 2 ppm or less than 1 ppm. Said another way, the difference between the concentration of nitric oxide in a first pulse and the concentration of nitric oxide in a second pulse is less than 10 ppm, less than 5 ppm, less than 2 ppm or less than 1 ppm.

The system was delivering 20 ppm of NO in 21% oxygen using an infant ventilator (Bio-Med Devices CV2+) with the ventilator settings shown in Table 1. The slower breathing rate was used as the worst case for NO mixing, because of the longer pause during exhalation.

TABLE 1
Ventilator Settings
Ventilator Settings

| | Pressure |
|---|---|
| Mode | Control |
| Rate (BPM) | 40 |
| Inspiratory Time INSP (sec) | 0.50 |
| Flow (LPM) | 6.0 |
| I:E Ratio | 1:2.0 |

The NO measurements were within product specifications (±20%). The conversion of NO₂ to NO in the cartridge overcomes the formation of NO₂ that is caused by the delay due to mixing.

As discussed above, the mixing can occur if the volume of the cartridge exceeds the ventilator pulse volume. For example, a 6000 ml/min and 40 breaths per minute the volume of the pulse is 150 ml. Good mixing can occur as long as the volume of the mixing chamber is greater than twice this volume.

The cartridge can converts essentially all of the NO₂ that was formed back into NO. These two figures clearly demonstrate the effect of a cartridge for converting NO₂ into NO, namely the cartridge reduced the NO₂ level as measured at the patient from 0.6 to 0 ppm.

The mixing performance of the cartridge was assessed using a high speed chemiluminescence detector with a 90% rise time of 250 msec. A very high speed NO detector was needed to catch the intra-breath variability of nitric oxide.

Previous technology partially solved this problem by tracking the rapid intra-breath flow changes in the ventilator circuit and uses the electronic signal from the flow sensor to synchronize the valve that introduces the NO into the circuit. This is a difficult and complex electronic solution that requires high speed sensors and very fast computer algorithms operating in real time. Because it is so difficult to execute, the FDA (in their Guidance document) allows the NO to vary from 0 to 150% of the mean, if the total duration of these transient concentrations did not exceed 10% of the volumetric duration of the breath.

Ideal mixing can happen when the NO gas is premixed and delivered directly using the ventilator. This perfect mixing condition can provide a baseline in order to validate chemiluminescence measurements under pulsing conditions. A blender was used to premix 800 ppm of NO with air to generate a 20 ppm gas to be delivered using a ventilator only. Chemiluminescence was used to measure the NO delivered to the artificial lung. FIG. 8 shows the results. From the peaks in the NO plot (top), it is evident that the chemiluminescence device was affected by the pulsing nature of the flow (bottom). The NO measurements were almost flat but some variations were still present.

Constant NO injection into the breathing circuit can be a simple and viable technique as long as a cartridge is both a mixer with sufficient volume and can remove NO₂ from the circuit or can convert the NO₂ back into NO.

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

What is claimed:

1. A portable nitric oxide delivery system comprising:
a disposable subsystem including
a nitric oxide-releasing agent;
a packaged cassette containing a reactor cartridge;
a reservoir containing a nitric oxide-releasing agent and configured to release the nitric oxide-releasing agent into the reactor cartridge;
a reactor cartridge containing a reducing agent that coverts a nitric oxide-releasing agent to nitric oxide (NO);
a reusable subsystem including
a portable console configured to receive the cassette; and
a respiratory assist device configured to deliver the NO to a subject.

2. The system of claim 1 further comprising an air pump configured to provide air flow to the reactor cartridge, such that a mixture of air and NO is delivered to a patient.

3. The system of claim 1 further comprising a pressure sensor.

4. The system of claim 1 wherein the reservoir contains a fixed volume of liquid dinitrogen tetroxide (N₂O₄), which is in equilibrium with NO₂ gas.

5. The system of claim 1 further comprising a nasal cannula configured to deliver the NO.

6. The system of claim 5 further comprising a nasal piece at end of cannula.

7. The system of claim 1 wherein the respiratory assist device is an oxygenator.

8. The system of claim 1, wherein the cartridge is disposable.

9. The system of claim 1 further comprising an additional cartridge.

10. The system of claim 7, wherein the additional cartridge is disposable.

11. The system of claim 7, wherein the first and second cartridges are identical twin cartridges.

12. The system of claim 1 further comprising a third cartridge in the gas line to the patient.

13. The system of claim 1 wherein the reservoir includes glass vial.

14. The system of claim 1 wherein the reservoir includes a sealed metal tube.

15. The system of claim 1 wherein the reservoir is a glass vial in a sealed metal tube

16. The system of claim 1 wherein the system is battery operated.

17. The system of of claim 1 wherein the nitric oxide-releasing agent is nitrogen dioxide (NO₂).

18. The system of claim 1 wherein the nitric oxide-releasing agent is dinitrogen tetroxide (N₂O₄).

19. The system of claim 1 wherein the nitric oxide-releasing agent is a nitrite ion (NO₂⁻).

20. The system of claim 1 wherein the reducing agent is ascorbic acid.

21. A method for delivering NO to a subject comprising
providing a nitric oxide-releasing agent;
providing a reactor cartridge containing a reducing agent that coverts the nitric oxide-releasing agent to nitric oxide (NO) in a packaged cassette;
inserting the packaged cassette into a portable console;
causing the nitric oxide-releasing agent within the reactor cartridge to release a nitric oxide-releasing agent;
causing a reducing agent within the reactor cartridge to convert a nitric oxide-releasing agent to nitric oxide (NO);
flowing the NO through a respiratory assist device configured to deliver the NO to a subject.

22. The method of claim 21 further comprising applying implantable heart pressure sensors to control the amount of inhaled NO delivered to a patient in need of NO.

23. The method of claim 22 further comprising applying a pulse oximeter to control the amount of inhaled NO delivered to a patient in need of NO.

24. The method of claim 21, wherein the nitric oxide is delivered from a fixed delivery platform.

25. The method of claim 21, wherein the nitric oxide is delivered from a mobile delivery platform.

26. The method of claim 21, wherein the nitric oxide is delivered from a bedside delivery platform.

27. The method of claim 21, wherein liquid N₂O₄ is the source of NO.

28. The method of claim 21, wherein the nitric oxide-releasing agent is provided from air by electrical discharge.

29. The method of claim 21, wherein the nitric oxide-releasing agent is provided from a tank of compressed gas.

30. A method for delivering NO to a subject comprising administering inhaled NO to a patient, and controlling and maintaining an optimum metabolic concentration of administered NO by feedback loop in real time.

31. The method of claim 30, wherein the feedback loop is controlled by an implantable right heart pressure sensor to maintain optimum metabolic concentration.

32. The method of claim 30, wherein the feedback loop is controlled by a pulse oximeter to maintain optimum metabolic concentration.

33. The method of claim 30, wherein controlling the concentration includes receiving patient data and using the data to calculate an optimum concentration to be delivered to the patient in real time.

34. The method of claim 30, wherein the nitric oxide is delivered from a fixed delivery platform.

35. The method of claim 30, wherein the nitric oxide is delivered from a mobile delivery platform.

36. The method of claim 30, wherein the nitric oxide is delivered from a bedside delivery platform.

37. The method of claim 30, wherein liquid N₂O₄ is the source of NO.

38. The method of claim 30, wherein the nitric oxide-releasing agent is provided from air by electrical discharge.

39. The method of claim 30, wherein the nitric oxide-releasing agent is provided from a tank of compressed gas.
